(19) United States
(12) Patent Application Publication
(10) Pub. No.: US 2025/0360052 A1
(43) Pub. Date: Nov. 27, 2025

(54) FLEXIBLE ELECTRODE FOR ACUPUNCTURE AND METHOD FOR MANUFACTURING SAME

(71) Applicant: SHANGHAI STAIRMED TECHNOLOGY CO., LTD., Shanghai (CN)

(72) Inventors: Xue LI, SHANGHAI (CN); Zhengtuo ZHAO, SHANGHAI (CN); Xiaocheng LI, SHANGHAI (CN)

(21) Appl. No.: 18/874,997

(22) PCT Filed: Jun. 29, 2022
(86) PCT No.: PCT/CN2022/102189

§ 371 Date: Dec. 13, 2024

(30) Foreign Application Priority Data
Jun. 17, 2022 (CN) 202210688562.4

(51) Int. Cl. A61H 39/00; A61H 39/08; A61N 1/05; A61N 1/375

(52) U.S. Cl. CPC A61H 39/002; A61H 39/08; A61N 1/0502; A61N 1/375

(57) ABSTRACT

A flexible electrode (100, 200) for acupuncture and a method (300) for manufacturing the same. An implanted portion of the flexible electrode (100, 200) can be implanted into an acupoint for a long or short term. The flexible electrode (100, 200) includes a first insulating layer (201), a second insulating layer (202), and a wire layer (203) located between the first and second insulating layers (201, 202). The implanted portion (110, 210) includes multiple electrode sites, each electrode site is electrically coupled to one of multiple wires in the wire layer (203), and is in contact with the acupoint after implantation. The multiple wires are configured to respectively apply identical or different electrical stimulations to the acupoint through the multiple electrode sites after implantation.

### CROSS-REFERENCE TO RELATED APPLICATIONS

This application is based on and claims the priority to the Chinese application No. 202210688562.4, filed on Jun. 17, 2022, the disclosure of which is hereby incorporated into the present application by reference in its entirety.

### TECHNICAL FIELD

The present disclosure relates to the technology field of life science, and more particularly, to a flexible electrode for acupuncture and a method for manufacturing the same.

### BACKGROUND

At present, the needle used for acupuncture treatment includes the filiform needle, the wide needle, the three-edged needle and the like, made of high-quality steel or alloy, and the basic structure thereof is generally divided into three parts: the needle handle, the needle body and the needle tip. Electroacupuncture applies a pulse power on the needle body of the above needle to realize the current stimulation to one acupoint or among a plurality of acupoints, so as to act on the patient's body meridians and collaterals to achieve the therapeutic effect.

### SUMMARY

A brief overview of the present disclosure is given below in order to provide a basic understanding of some aspects of the present disclosure. However, it should be understood that this overview is not an exhaustive overview of the present disclosure. It is not intended to identify the key or important part of the present disclosure, nor to limit the scope of the present disclosure. Its purpose is simply to give some concepts of the present disclosure in a simplified form as a prelude to a more detailed description given later.

According to a first aspect of the present disclosure, a flexible electrode for acupuncture is provided. An implanted portion of the flexible electrode is able to be implanted into an acupoint for a long or short term, wherein the flexible electrode includes a first insulating layer, a second insulating layer, and a wire layer located between the first insulating layer and the second insulating layer; the implanted portion includes a plurality of electrode sites, each electrode site is electrically coupled to one of a plurality of wires in the wire layer, and is in contact with the acupoint after implantation; and the plurality of wires are to respectively apply identical or different electrical stimulations to the acupoint through the plurality of electrode sites after implantation.

According to a second aspect of the present disclosure, a method for manufacturing the flexible electrode for acupuncture according to the first aspect of the present disclosure is provided. The method includes: manufacturing the first insulating layer, the wire layer, the second insulating layer, and the electrode site on a substrate; and separating the flexible electrode from the substrate, wherein a through-hole is manufactured by patterning at a position corresponding to the electrode site in at least one of the first insulating layer and the second insulating layer.

Other features and their advantages of the present disclosure will become more apparent from the following detailed description of exemplary embodiments of the present disclosure with reference to the accompanying drawings.

### BRIEF DESCRIPTION OF THE DRAWINGS

The accompanying drawings, which constitute a part of the specification, describe the embodiments of the present disclosure and, together with the description, serve to explain the principle of the present disclosure.

The present disclosure may be more clearly understood from the following detailed description with reference to the accompanying drawings, in which:

FIG. 1 shows a schematic diagram of at least a portion of a flexible electrode for acupuncture according to an embodiment of the present disclosure;

FIG. 2 shows an exploded diagram of at least a portion of a flexible electrode for acupuncture according to an embodiment of the present disclosure;

FIG. 3 shows a flowchart of a method for manufacturing a flexible electrode for acupuncture according to an embodiment of the present disclosure; and

FIG. 4 shows a schematic diagram of a method for manufacturing a flexible electrode for acupuncture according to an embodiment of the present disclosure.

### DETAILED DESCRIPTION

The following detailed description is made with reference to the accompanying drawings, and the following detailed description is provided to assist in a comprehensive understanding of the various exemplary embodiments of the present disclosure. The following description includes various details to assist in understanding, but these details are considered to be examples only and not to limit the present disclosure, and the present disclosure is defined by the appended claims and their equivalents. The words and phrases used in the following description are only used to enable a clear and consistent understanding of the present disclosure. In addition, for clarity and brevity, descriptions of well-known structures, functions, and configurations may be omitted. Those of ordinary skill in the art will recognize that various changes and modifications may be made to the examples described herein without departing from the spirit and scope of the present disclosure.

The following description of at least one exemplary embodiment is in fact merely illustrative and is in no way intended to limit the present disclosure and its application or usage. That is, the structures and methods herein are shown in an exemplary manner to illustrate different embodiments of the structures and methods in the present disclosure. However, those skilled in the art will appreciate that they merely illustrate exemplary ways of the present disclosure that can be implemented, rather than in exhaustive ways. In addition, the drawings need not be drawn to scale, and some features may be enlarged to illustrate the details of the specific components.

Technologies, methods, and apparatus known to ordinary technicians in the relevant field may not be discussed in detail, but where appropriate, such technologies, methods, and apparatus should be considered as a part of the granted specification.

In all examples shown and discussed herein, any specific value should be interpreted as merely exemplary and not as limiting. Therefore, other examples of the exemplary embodiments may have different values.

Existing needles for electroacupuncture generally include the following types. One type of the needle for electroacupuncture includes the positive and negative electrodes in a shape of sheet with a pattern made of conductive rubber. The two positive and negative electrodes are respectively adhered on two different acupoints, and perform the electric pulse stimulation on the human acupoints after the power supply is turned on, so as to generate the therapeutic effect. This adhesive electrode has a large area, leading to an inaccurate selection of the acupoint; and it can only perform the electric pulse stimulation on the skin surface of a human, but cannot go deep into the human body, and is difficult to obtain a good therapeutic effect. Another type of the needle for electroacupuncture includes a filiform needle made of traditional stainless steel. The filiform needles are penetrated into two acupoints and perform the electric pulse stimulation on the human body acupoints after the power supply is turned on. In this design, the needles must be penetrated into two acupoints, and be respectively connected to the positive and negative electrodes to form the current path and to generate the electric pulse stimulation. Still another type of the needle for electroacupuncture includes at least two needle bodies, one of which is a positive needle body and the other is a negative needle body. The positive and negative needle bodies are separated by an insulating material body and connected as a whole, and the positive and negative needle bodies are respectively connected with the positive and negative power wires.

The disadvantages of these needles for electroacupuncture include: the low-frequency pulse current passing through the needle body is used for stimulating the acupoint to make the ions in the human tissue move for electroacupuncture treatment, but it is impossible to accurately control and determine the direction of the pulse current, and there is a certain degree of blindness; the current is evenly distributed from the needle body to the needle tip within the human tissue, and the small current pulse cannot be completely concentrated at the needle tip, resulting in the poor therapeutic effect; the electric current flows between the needle body and the human tissue, causing the unnecessary stimulation or even the damage to the non-targeted subcutaneous cells, tissue, or organ, and the current on the needle body may further electrolyze the tissue fluid, causing the corrosion to the needle body; and the needle sensation is closely related to the analgesic effect of acupuncture, as the needle sensation disappears, the analgesic effect of acupuncture also disappears, so the duration of needle sensation is positively correlated with the therapeutic effect, however, the metal needle body cannot be left in the body for a long period of time, and cannot achieve the effective retention duration of needle of long-term stimulation on the acupoint.

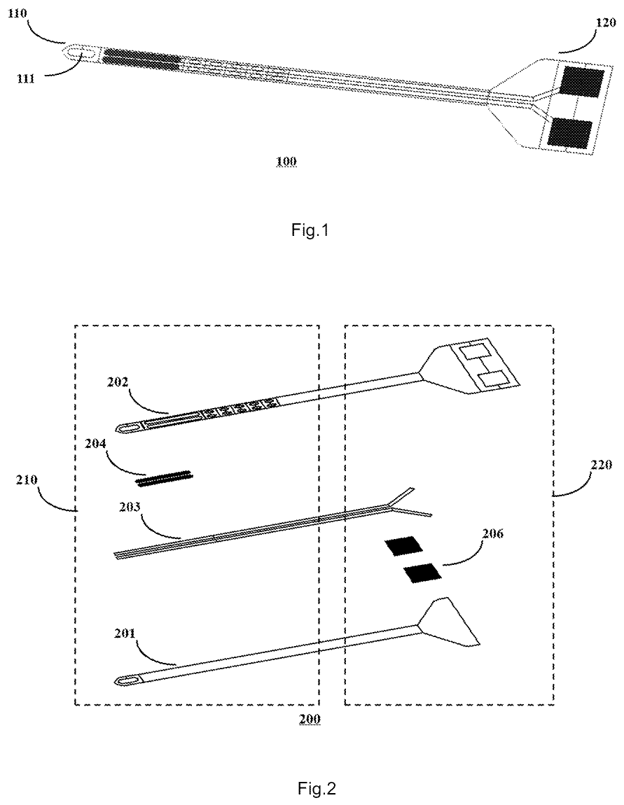

FIG. 1 shows a schematic diagram of at least a portion of a flexible electrode 100 for acupuncture according to an embodiment of the present disclosure. As shown in FIG. 1, the flexible electrode 100 may include an implanted portion 110, which may be implanted into an acupoint for a long or short term, so as to further apply an electrical stimulation to the acupoint while realizing the acupuncture effect for the acupoint. In an embodiment according to the present disclosure, the implanted portion 110 may further have an installation hole 111, through which an electrode implantation device may be attached to the flexible electrode 100 and drive the flexible electrode 100 during implantation, thereby guiding the flexible electrode to complete the implantation process. The flexible electrode 100 shown in FIG. 1 includes the implanted portion 110 with an elongated needle shape. However, it should be understood that FIG. 1 only shows a non-limiting example, and the flexible electrode for acupuncture may be provided with the implanted portion 110 with a different shape and size as required. The flexible electrode 100 may further include a rear end portion 120, which may be used for engaging the flexible electrode 100 and a rear end circuit for rear end adaptation. The implanted portion 110 can extend from the rear end portion 120.

FIG. 2 shows an exploded diagram of at least a portion of a flexible electrode 200 for acupuncture according to an embodiment of the present disclosure. It can be clearly seen from FIG. 2 that the flexible electrode 200 has a multi-layer structure. Specifically, the flexible electrode 200 includes a bottom insulating layer 201, a top insulating layer 202, a wire layer 203, an electrode site layer 204, a rear end site layer 206 and the like. It should be understood that various layers of the flexible electrode 200 shown in FIG. 2 are only non-limiting examples, and the flexible electrode in the present disclosure may omit one or more of these layers, or may include more other layers.

The flexible electrode 200 may include an insulating layer 201 at the bottom and an insulating layer 202 at the top. Specifically, as shown in FIG. 2, the implanted portion 210 and the rear end portion 220 of the flexible electrode 200 may both include the insulating layers 201 and 202. The insulating layer in the flexible electrode may refer to an outer surface layer that plays an insulating role in the electrode. Since the insulating layer of the flexible electrode needs to be in contact with a biological tissue after implantation, a material of the insulating layer is required to have a good insulation property and at the same time a good biocompatibility. In an embodiment of the present disclosure, the material of the insulating layers 201 and 202 may include polyimide (PI), polydimethylsiloxane (PDMS), Parylene, epoxy resin, polyamide imide (PAI), SU-8 photoresist, silica gel, silicone rubber, and the like. In an embodiment according to the present disclosure, in order to make the flexible electrode further have a biodegradable property, the material of the insulating layers 201 and 202 may include polylactic acid, polylactic acid-glycolic acid copolymer, and the like. In addition, the insulating layers 201 and 202 are further main portions that provide the strength in the flexible electrode 200. If the insulating layer is too thin, the strength of the electrode will be reduced; and if the insulating layer is too thick, the flexibility of the electrode will be reduced. In addition, the implantation of the electrode including the overly thick insulating layers will cause a relatively great damage to the organism. In an embodiment according to the present disclosure, the thickness of the insulating layers 201 and 202 may be 0.5 μm to 1 mm.

The flexible electrode 200 may further include a wire in the wire layer 203 between the bottom insulating layer 201 and the top insulating layer 202. Specifically, as shown in FIG. 2, the implanted portion 210 and the rear end portion 220 of the flexible electrode 200 may both include the wire layer 203, and the wire in the wire layer 203 extend from the rear end portion 220 to the implanted portion 210. In an embodiment according to the present disclosure, the flexible electrode 200 may include a plurality of wires in a same wire layer 203, wherein each wire may be electrically coupled to one of the electrode sites in the electrode site layer 204 and be electrically coupled to one of the rear end sites in the rear end site layer 206, so that the electrical stimulation signal received at the rear end site is applied to the electrode site implanted at the acupoint through the wire. The wire layer 203 shown in FIG. 2 includes two wires extending from the rear end portion 220 to the implanted portion 210, but it should be understood that the number of wires in the flexible electrode is not limited thereto. In an embodiment according to the present disclosure, a cross-sectional area of each wire in the wire layer 203 may be 0.01 μm² to 1 mm². It should be understood that the size of the wire is not limited to the above-listed range, but may vary according to the design requirement.

In an embodiment according to the present disclosure, the wire in the wire layer 203 may have a film structure including a plurality of sub-layers stacked in the thickness direction. A material of these sub-layers may be a material that can enhance the adhesion, ductility, conductivity, and the like of the wire. As a non-limiting example, the wire layer 203 may be a metal film including three sub-layers stacked, wherein the first sub-layer and the second sub-layer that are in contact with the insulating layers 201 and 202 respectively, are adhesion sub-layers, and a metal or non-metal adhesive material such as titanium (Ti), titanium nitride (TiN), chromium (Cr), tantalum (Ta), tantalum nitride (TaN) or the like may be adopted; and the third sub-layer located between the first sub-layer and the second sub-layer is a conductive sub-layer, and a material with good conductivity such as gold (Au), platinum (Pt), iridium (Ir), tungsten (W), platinum-iridium alloy, titanium alloy, graphite, carbon nanotubes, PEDOT or the like may be adopted. In an embodiment according to the present disclosure, in order to make the flexible electrode further have the biodegradable property, the conductive sub-layer may adopt a material such as magnesium (Mg), molybdenum (Mo), the alloy thereof or the like. It should be understood that the wire layer may be made of another conductive metal or non-metal material, or may be made of a conductive polymeric material and a conductive composite material. In an embodiment according to the present disclosure, the thickness of the adhesion sub-layer may be 1nm to 50nm, and the thickness of the conductive sub-layer may be 5nm to 200 μm.

As shown in FIG. 2, the implanted portion 210 of the flexible electrode 200 may further include electrode sites located in the top electrode site layer 204. Each electrode site is electrically coupled to one of the wires in the wire layer 203, and contacts the acupoint after implantation of the flexible electrode 200 to apply the electrical stimulation to the acupoint. The electrode site layer 204 of the flexible electrode 200 shown in FIG. 2 is located between the top insulating layer 202 and the wire layer 203, and the electrode sites in the electrode site layer 204 are exposed to the outer surface of the flexible electrode 200 via the through-holes in the top insulating layer 202. After the flexible electrode is implanted, such a design enables the electrode sites of the flexible electrode to contact the acupoint into which the flexible electrode is implanted, and the electrode sites do not have to be located at the outermost side of the flexible electrode, so that the electrode sites are not easy to fall off from the flexible electrode after implantation, which is conducive to a long-term and stable application of the electrical stimulation. In an embodiment according to the present disclosure, the flexible electrode may not include a separate electrode site layer. In this case, the electrode site may be located in the wire layer 203, electrically coupled to the corresponding wire in the wire layer 203 (for example, through the metal trace in the wire layer 203), and exposed to the outer surface of the flexible electrode 200 via the through-hole in at least one of the bottom insulating layer 201 and the top insulating layer 202, and contact the acupoint into which the flexible electrode 200 is implanted. In an embodiment according to the present disclosure, the flexible electrode 200 may further include an electrode site in the electrode site layer 204 and an electrode site in the wire layer 203 at the same time, and the electrode sites in the electrode site layer 204 and in the wire layer 203 are both exposed to the outer surface of the flexible electrode 200 via the through-holes in at least one of the bottom insulating layer 201 and the top insulating layer 202, and contact the acupoint into which the flexible electrode 200 is implanted. The flexible electrode 200 shown in FIG. 2 includes two wires and two electrode sites in the electrode site layer 204 that are respectively coupled to these two wires. However, it should be understood that the present disclosure is not limited thereto, but may include more electrode sites.

In an embodiment according to the present disclosure, in the case where the flexible electrode 200 includes the electrode site layer 204, the electrode site in the electrode site layer 204 may have a film structure including a plurality of sub-layers stacked in the thickness direction. The material of the adhesion sub-layer close to the wire layer 203 in the plurality of sub-layers may be a material that can enhance the adhesion between the electrode site and the wire, and the thickness of the adhesion sub-layer may be 1 nm to 50 nm. As a non-limiting example, the electrode site layer 204 may be a metal film including two stacked sub-layers, wherein the first sub-layer close to the wire layer 203 is Ti, TiN, Cr, Ta or TaN, and the second sub-layer of the electrode site layer 204 exposed to outside is Au. It should be understood that the electrode site layer may be similar to the wire layer and may be made of another conductive metal or non-metal material, such as Pt, Ir, W, Mg, Mo, platinum-iridium alloy, titanium alloy, graphite, carbon nanotubes, PEDOT, or the like.

Each electrode site may have a planar size of micrometer level and a thickness of nanometer level. The electrode site in the electrode site layer 204 shown in FIG. 2 has an elongated shape, and its length occupies a considerable portion of the entire length of the implanted portion 210, so as to fully apply the electrical stimulation at the acupoint where implantation is performed, but it should be understood that the shape of the electrode site is not limited thereto. In an embodiment according to the present disclosure, the electrode site may have various regular or irregular shapes configured as required, the number of 2 to 2000, the maximum side length or diameter of 1 μm to 2 mm, the interval from another electrode site of 10μm to 10 mm, and the thickness of 5nm to 200 μm. It should be understood that the shape, number, size and interval of the electrode site may be selected as required.

In an embodiment according to the present disclosure, the surface of the electrode site exposed to outside and in contact with the acupoint may further have a surface modification layer to improve an electrochemical property of the electrode site. As a non-limiting example, the surface modification layer may be obtained by using electro-induced polymerization coating of PEDOT: PSS, sputtering of iridium oxide film, and the like, so as to reduce the impedance and to enhance the charge input capacity of the electrode, thereby increasing the input current of the electrode and increasing the stability of the flexible electrode when applying the stimulation.

In an embodiment according to the present disclosure, the flexible electrode may include a plurality of electrode site layers. Although not shown in FIG. 2, the flexible electrode 200 may further include electrode sites in a bottom electrode site layer 205 between the bottom insulating layer 201 and the wire layer 203, and these electrode sites may be exposed to an outer surface of the flexible electrode 200 via the through-holes in the bottom insulating layer 201, and may contact the acupoint to apply electrical stimulation signals after the flexible electrode is implanted. Similar to the electrode sites in the top electrode site layer 204, in the flexible electrode 200, each electrode site in the bottom electrode site layer 205 may be electrically coupled to one of the wires in the wire layer 203. In an embodiment according to the present disclosure, the electrode sites in the bottom electrode site layer 205 and the electrode sites in the top electrode site layer 204 may be located at opposite positions on two sides of the wire layer 203 of the flexible electrode 200, and the electrode sites in the bottom and top electrode site layers 205 and 204 located at the opposite positions may be electrically coupled to a same wire in the wire layer 203. In an embodiment according to the present disclosure, the electrode sites in the bottom electrode site layer 205 and the electrode sites in the top electrode site layer 204 may be located at different positions on two sides of the wire layer 203 of the flexible electrode 200, so as to apply electrical stimulation signals at different positions (for example, at different depths) of the acupoint, and the electrode site in the bottom electrode site layer 205 may be electrically coupled to a wire in the wire layer 203 other than the wire electrically coupled to the electrode site in the top electrode site layer 204.

It should be understood that the bottom electrode site layer 205 is an optional part instead of a necessary part of the flexible electrode. For example, the flexible electrode in the present disclosure may only include the top electrode site layer 204 and may not include the bottom electrode site layer 205. The shape, size, material, and the like of the bottom electrode site may be similar to those of the top electrode site and will not be described in detail here.

In an embodiment of the present disclosure, the flexible electrode may further include an additional wire layer, that is, the flexible electrode in the present disclosure may include one or more wire layers. The size, material, manufacturing method, and the like of the additional wire layer may be similar to those of the wire layer 203, and will not be described in detail here. In the case where the flexible electrode includes the additional wire layer, these wire layers may be separated by an additional insulating layer, and the size, material, and manufacturing method of the additional insulating layer may be similar to those of the bottom insulating layer 201 and/or the top insulating layer 202, and will not be described in detail here. One or more wires in these additional wire layers may be electrically coupled to the electrode site(s) of the flexible electrode. By including a plurality of wire layers in the flexible electrode, the number and accuracy of the signals transmitted through the flexible electrode can be increased.

The rear end portion 220 of the flexible electrode 200 may include rear end sites in the rear end site layer 206, wherein each rear end site in the rear end site layer 206 may be electrically coupled to one of the wires in the wire layer 203 and electrically coupled to the rear end circuit via a through-hole in the top insulating layer 202, so as to achieve the signal transmission between the electrode site electrically coupled to the wire and the rear end circuit. As shown in FIG. 2, the rear end site layer 206 is located between the wire layer 203 and the bottom insulating layer 201, and the rear end sites in the rear end site layer 206 may be electrically coupled to the rear end circuit via through-holes in the top insulating layer 202. In an embodiment according to the present disclosure, the rear end site layer 206 may be located between the wire layer 203 and the top insulating layer 202, and the rear end sites in the rear end site layer may be exposed to the outer surface of the flexible electrode via through-holes in at least one of the top insulating layer 202 and the bottom insulating layer 201 and may be electrically coupled to the rear end circuit. In an embodiment according to the present disclosure, the flexible electrode may not include a separate rear end site layer. In this case, the rear end site may be located in the wire layer 203, electrically coupled to the corresponding wire in the wire layer 203, and exposed to the outer surface of the flexible electrode 200 via a through-hole in at least one of the bottom insulating layer 201 and the top insulating layer 202 and may be electrically coupled to the rear end circuit. Here, the rear end circuit may refer to a circuit at the rear end of the flexible electrode, such as a power supply, a pulse generator, a signal processing circuit, and the like, associated with the signal to be applied by the flexible electrode. The rear end site may have a planar size of micrometer level and a thickness of nanometer level. As a non-limiting example, the rear end site may be the BGA package site with a diameter of 50 μm to 2000 μm, or may be a circular, elliptical, rectangular, rounded rectangular, chamfered rectangular site with a side length of 50 μm to 2000 μm, and the thickness of the rear end site layer 206 and the rear end site therein may be 5 nm to 200 μm. It should be understood that the shape, size, and the like of the rear end site are not limited to the ranges listed above, but may vary according to the design requirement.

In an embodiment according to the present disclosure, the rear end site in connection manner may include a plurality of sub-layers in the thickness direction, the material of the adhesion sub-layer of the plurality of sub-layers close to the wire layer 203 may be a material that can enhance the adhesion between the rear end site and the wire; the material of the middle flux sub-layer of the plurality of sub-layers may be a flux material; the material of the conductive sub-layer of the plurality of sub-layers may be other conductive metal or non-metallic material such as that of the wire layer mentioned above; and the outermost layers of the plurality of sub-layers that may be exposed through the insulating layers 201 and 202 is anti-oxidation protective sub-layers. As a non-limiting example, the rear end site layer 206 may be a metal film including three superimposed sub-layers, wherein the first sub-layer close to the wire layer 203 may be the adhesion sub-layer in nanometer-scale to improve the adhesion between the rear end site layer 206 and the wire layer 203. The material of the first sub-layer as the adhesion sub-layer may be Cr, Ta, TaN, Ti, TiN, or the like; the material of the second sub-layer as the flux sub-layer may be nickel (Ni), Pt or palladium (Pd); and the material of the third layer as the conductive sub-layer may be Au, Pt, Ir, W, Mg, Mo, platinum-iridium alloy, titanium alloy, graphite, carbon nanotubes, PEDOT, or the like. It should be understood that the rear end site layer may be made of other conductive metal or non-metallic material. The rear end site layer 206 in FIG. 2 is a part connected to a rear end processing system or chip, and the design of the size, interval, shape, and the like of the site therein may vary according to different connection manner of the rear end.

In an embodiment according to the present disclosure, the flexible electrode may not include the site layer such as the top electrode site layer, the bottom electrode site layer, the rear end site layer, or the like. In this case, the electrode site in the flexible electrode for applying electrical stimulation and the rear end site in the rear end portion for adaptation may both be parts of the wire layer, and electrically coupled to corresponding wires in the wire layer. Furthermore, the electrode site for sensing and applying electrical signal may directly contact the tissue area into which the electrode is implanted. As a non-limiting example, each electrode site may be electrically coupled in the wire layer to a corresponding wire in the wire layer, and exposed to the outer surface of the electrode via a corresponding through-hole in the top insulating layer or the bottom insulating layer and in contact with a biological tissue.

In an embodiment according to the present disclosure, after the flexible electrode 200 is separated from the substrate, the rear end portion 220 of the flexible electrode 200 may be connected to the rear end circuit, and the flexible electrode 200 and the rear end circuit connected to the rear end portion 220 may be packaged together by any one of epoxy resin and polydimethylsiloxane, or a combination thereof, to improve the connection strength between the flexible electrode 200 and the rear end circuit.

When the flexible electrode disclosed herein is applied for acupuncture stimulation, the rear end circuit (such as a pulse generating device) may provide stimulation signal(s) to apply identical or different electrical stimulations to the acupoint through a plurality of electrode sites, and the stimulation signal is transmitted to the electrode site by the rear end site via the wire. The identical or different electrical stimulations may be applied for a long or short term, may be current(s) or voltage(s) with identical or different polarities, may be current(s) or voltage(s) with identical or different amplitudes, wave widths, and frequencies. Therefore, the flexible electrode according to the present disclosure can realize the asynchronous multi-current channel stimulation to a single acupoint.

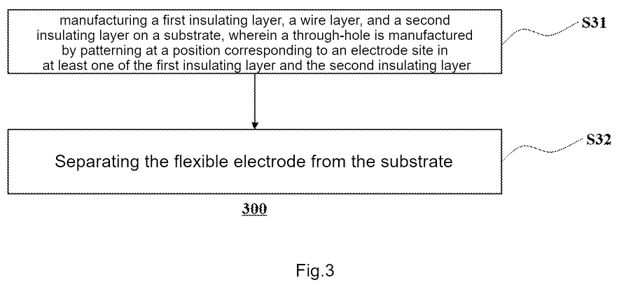

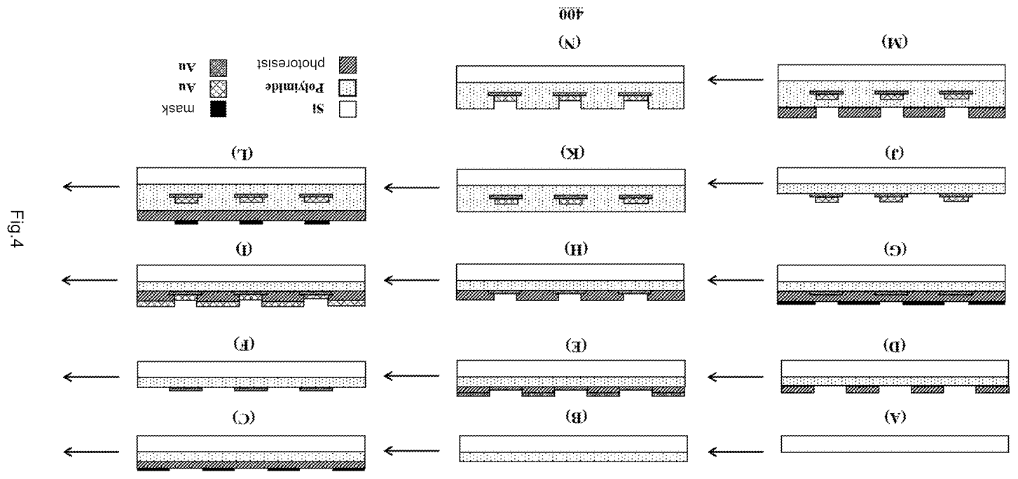

FIG. 3 shows a flowchart of a method 300 for manufacturing a flexible electrode for acupuncture according to an embodiment of the present disclosure. In the present disclosure, the manufacturing method based on a micro-electro mechanical system (MEMS) process may be adopted to manufacture a nano-scale flexible electrode. As shown in FIG. 3, the method 300 may include: at S31, manufacturing a first insulating layer, a wire layer, and a second insulating layer on a substrate, wherein a through-hole is manufactured by patterning at a position corresponding to the electrode site in at least one of the first insulating layer and the second insulating layer; and at S32, separating the flexible electrode from the substrate. The steps for manufacturing various layers of the flexible electrode at S31 are described in detail below in conjunction with FIG. 4.

FIG. 4 shows a schematic diagram of a method 400 for manufacturing a flexible electrode for acupuncture according to an embodiment of the present disclosure, wherein the flexible electrode for acupuncture may be the flexible electrodes 100 and 200 shown in FIGS. 1 and 2. The manufacturing process and structure of the bottom insulating layer, the wire layer, the electrode site layer, the top insulating layer, and the like of the flexible electrode are described in more detail in conjunction with FIG. 4.

The view (A) of FIG. 4 shows the substrate of the electrode. In an embodiment according to the present disclosure, a hard substrate such as glass, quartz, silicon wafer, or the like, may be adopted. In an embodiment of the present disclosure, another soft material may be adopted as the substrate, such as the same material as that of the insulating layer.

The view (B) of FIG. 4 shows manufacturing of the bottom insulating layer on the substrate. As a non-limiting example, in the case where the insulating layer adopts the polyimide material, the manufacturing of the bottom insulating layer may include steps of a film forming process, a formed-film curing, an enhanced curing and the like to form a film as the insulating layer. The film forming process may include coating polyimide on the substrate, for example, a layer of polyimide may be spin coated in segmented rotational speeds. The formed-film curing may include gradually heating to a relatively high temperature and keeping the high temperature to form a film, so as to perform a subsequent processing step. The enhanced curing may include multi-gradient temperature increasing before manufacturing of a subsequent layer, preferably heating in a vacuum or nitrogen atmosphere and baking for several hours. It should be understood that the above manufacturing process is only a non-limiting example of the manufacturing process of the bottom insulating layer, and one or more of the steps may be omitted, or more other steps may be included.

Views (C) to (F) of FIG. 4 show manufacturing of the wire layer on the bottom insulating layer. As shown in the view (C), photoresist and a mask may be applied on the bottom insulating layer. It should be understood that another photolithography manner may be adopted to prepare the patterned film, such as laser direct writing, electron beam photolithography and the like. In an embodiment according to the present disclosure, for a metal film such as the wire layer, double layers of photoresist may be applied to facilitate the manufacturing (evaporation or sputtering) and lifting off of the patterned film. By providing the pattern of the mask associated with the wire layer, for example, the pattern of the wire layer described above may be achieved, such as the pattern of the wire layer 203 of FIG. 2. Next, exposure and development may be performed to obtain a structure as shown in the view (D). In an embodiment according to the present disclosure, the exposure may be carried out by contact photolithography, wherein the mask and the structure are exposed in a vacuum contact mode. This step may further include the alignment between the layers. Next, a film may be formed on the structure shown in the view (D), for example, a process such as evaporation, sputtering or the like may be used for depositing a metal film material, such as Au, to obtain a structure shown in the view (E). Next, lifting off may be performed to separate the film in the non-patterned area from the film in the patterned area by removing the photoresist in the non-patterned area, so as to obtain a structure as shown in the view (F), that is, to form the wire layer. In an embodiment according to the present disclosure, a photoresist removing treatment may be performed again after lifting off by photoresist removing, so as to further remove the residual photoresist on the surface of the structure.

In an embodiment according to the present disclosure, before manufacturing the wire layer, a rear end site layer may be formed. As a non-limiting example, the manufacturing process of the rear end site layer may be similar to that of the metal film described above with respect to the wire layer.

It should be noted that the above manufacturing process is directed to an embodiment of manufacturing a flexible electrode without a bottom electrode site layer or a through-hole corresponding to the electrode site in the bottom insulating layer. If the flexible electrode includes a bottom electrode site layer, the bottom electrode site layer may be manufactured on the bottom insulating layer before the wire layer is manufactured. The manufacturing steps of the bottom electrode site layer are similar to those of the top electrode site layer and will be described in detail later with respect to the top electrode site layer. Accordingly, in the case where the flexible electrode includes a bottom electrode site, in the process of manufacturing the bottom insulating layer, in addition to the above steps, a patterning step may further be included for etching a through-hole at a position corresponding to the bottom electrode site in the bottom insulating layer. The step of patterning the insulating layer will be described in detail later with respect to the top insulating layer.

Views (G) to (J) of FIG. 4 show the steps for manufacturing the top electrode site layer, which are similar to those for manufacturing the wire layer in views (C) to (F), and are not described in detail herein. In the view (G), by providing the pattern of the mask associated with the top electrode site layer, for example, the pattern of the top electrode site layer described above may be achieved, such as the pattern of the electrode site layer 204 of FIG. 2.

Views (K) to (N) of FIG. 4 show manufacturing of the top insulating layer. For the photosensitive film, patterning may be generally achieved directly through patterned exposure and development, while for the non-photosensitive material adopted in the insulating layer, patterning cannot be achieved by exposing and developing the material itself. As such, a sufficient thick patterned anti-etching layer may be manufactured on this layer, and then the film in the area not covered by the anti-etching layer is removed by dry etching (the anti-etching layer will be thinned at the same time, so it is necessary to ensure that the anti-etching layer is thick enough), and then the anti-etching layer is removed to achieve the patterning of the non-photosensitive layer. As a non-limiting example, when manufacturing the insulating layer, the photoresist may be adopted as the anti-etching layer. The manufacturing of the top insulating layer may include the steps such as a film forming process, formed-film curing, patterning, enhanced curing and the like, wherein the view (K) shows the structure obtained after the film-forming of the top insulating layer, the view (L) shows the application of the photoresist and the mask on the top insulating layer after film-forming, the view (M) shows the structure of the anti-etching layer obtained after the steps including exposure and development, and the view (N) shows the structure including the manufactured top insulating layer. The film forming process, formed-film curing and enhanced curing have been described in detail above with respect to the bottom insulating layer, and description therefor is omitted here for brevity. The patterning step may be performed after the formed-film curing, or after the enhanced curing. After the enhanced curing, the insulating layer has a stronger anti-etching ability. Specifically, in the view (L), a sufficient thick layer of photoresist is manufactured on the insulating layer through steps such as spin coating, baking and the like. By providing the pattern of the mask associated with the top insulating layer, for example, the pattern of the top insulating layer 202 shown in FIG. 2 may be realized, that is, the contours of the implanted portion 210 (especially the installation hole and the through-hole for the electrode site included in the implanted portion 210) and the rear end portion 220 (especially the through-hole for the rear end site included in the rear end portion 220). In the view (M), the pattern is transferred to the photoresist on the insulating layer through the steps such as exposure, development and the like to obtain the anti-etching layer, wherein the portion to be removed from the top insulating layer is exposed. The exposed portion of the top insulating layer may be removed by oxygen plasma etching, and after flood exposure, the remaining photoresist on the top insulating layer is removed with developer, acetone, or the like, to obtain the structure shown in the view (N).

In an embodiment according to the present disclosure, the top insulating layer may be subjected to an adhesion enhancement treatment before being manufactured, so as to improve the bonding force between the bottom insulating layer and the top insulating layer.

The present disclosure provides a flexible electrode for acupuncture and a method for manufacturing the same. The flexible electrode is placed in an acupoint for a long or short term for an acute stimulation or a long-term stimulation, and applies an electrical stimulation while playing the role of acupuncture, so the stimulation effect is enhanced combined with the meridian acupoint; a highly integrated stimulation electrode is manufactured by micro-nano processing technology, and the asynchronous multi-current channel stimulation can be achieved through the control of the pulse generator; only one acupoint needs to be selected to be inserted with the flexible electrode, there is no need to select two acupoints, and the stimulation position is relatively accurate; the current distribution of the stimulation electrode is relatively concentrated, the therapeutic effect is good, and the damage to another structure and tissue in the human body can be avoided as much as possible; and the flexible material with good biocompatibility is adopted, and the flexible electrode can be left at the acupoint in a safe and comfortable state, without obvious discomfort, allergic reaction and toxic side effect.

The words “front”, “rear”, “top”, “bottom”, “above”, “below”, and the like, if present, in the specification and claims are used for descriptive purposes and are not necessarily used to describe an invariant relative position. It should be understood that the words so used are interchangeable where appropriate, such that the embodiments of the present disclosure described herein, for example, are capable of operation in other orientations than those illustrated or otherwise described herein.

As used herein, the word “exemplary” means “used as an example, instance, or illustration” rather than serving as a “model” to be exactly copied. Any implementation described as an example herein is not necessarily to be construed as preferred or advantageous over other implementations. Moreover, the present disclosure is not limited by any stated or implied theory given in the above sections of technical field, background, summary, or

### DETAILED DESCRIPTION

As used herein, the term “substantially” is intended to include any minor variations due to design or manufacturing imperfections, device or component tolerances, environmental influences, and/or other factors. The term “substantially” also allows for deviations from a perfect or ideal situation due to parasitic effects, noise, and other practical considerations that may exist in actual implementations.

The terms “first”, “second” and the like may be used herein for reference purposes only and are not intended to be limiting. For example, the terms “first”, “second” and other such numerical terms referring to structures or elements do not imply a sequence or order unless the context clearly indicates otherwise.

It should also be understood that when the term “include/comprise” is used herein, it indicates the presence of the specified features, integers, steps, operations, units and/or components, but does not exclude the presence or addition of one or more other features, integers, steps, operations, units and/or components and/or their combinations.

As used herein, the term “and/or” includes any and all combinations of one or more of the associated listed items. The terms used herein are for the purpose of describing specific embodiments only and are not intended to limit the present disclosure. As used herein, the singular forms “a”, “an”, and “the” are also intended to include the plural forms, unless the context clearly indicates otherwise.

Those skilled in the art will appreciate that the boundaries between the above operations are merely illustrative. Multiple operations may be combined into a single operation, a single operation may be distributed in additional operations, and operations may be performed at least partially overlapping in time. Moreover, alternative embodiments may include multiple instances of a particular operation, and the operation order may be changed in other various embodiments. However, other modifications, variations, and replacements are also possible. Therefore, this specification and accompanying drawings should be considered illustrative, not restrictive.

Although some specific embodiments of the present disclosure have been described in detail by way of example, it should be understood by those skilled in the art that the above examples are for illustration only and are not intended to limit the scope of the present disclosure. The various embodiments disclosed herein may be combined in any manner without departing from the spirit and scope of the present disclosure. It should also be understood by those skilled in the art that various modifications may be made to the embodiments without departing from the scope and spirit of the present disclosure. The scope of the present disclosure is defined by the appended claims.

What is claimed is:

1. A flexible electrode for acupuncture, an implanted portion of the flexible electrode being able to be implanted into an acupoint for a long or short term, wherein
the flexible electrode comprises a first insulating layer, a second insulating layer, and a wire layer located between the first insulating layer and the second insulating layer;
the implanted portion comprises a plurality of electrode sites, each electrode site is electrically coupled to one of a plurality of wires in the wire layer, and is in contact with the acupoint after implantation; and
the plurality of wires are configured to respectively apply identical or different electrical stimulations to the acupoint through the plurality of electrode sites after implantation.

2. The flexible electrode according to claim 1, wherein the flexible electrode comprises a plurality of wire layers, the plurality of wire layers are separated from each other by an additional insulating layer, and each wire layer comprises a plurality of wires separated from each other.

3. The flexible electrode according to claim 1 or 2, wherein the electrode site is located between the wire layer and at least one layer of the first insulating layer and the second insulating layer, and is exposed via a through-hole in the at least one layer.

4. The flexible electrode according to claim 3, wherein the electrode site comprises a conductive sub-layer, and a material of the conductive sub-layer is any one of gold, platinum, iridium, tungsten, magnesium, molybdenum, platinum-iridium alloy, titanium alloy, graphite, carbon nanotubes, PEDOT, or a combination thereof.

5. The flexible electrode according to claim 4, wherein the electrode site further comprises an adhesion sub-layer close to the wire layer, the adhesion sub-layer adopts a material capable of enhancing adhesion between the electrode site and the wire layer.

6. The flexible electrode according to claim 1 or 2, wherein the electrode site is located in the wire layer and exposed via a through-hole in at least one layer of the first insulating layer and the second insulating layer.

7. The flexible electrode according to claim 1 or 2, wherein a shape of the electrode site is provided as needed, a number of the electrode sites is 2 to 2000, a maximum side length or diameter of the electrode site is 1 micron to 2 millimeters, an interval from another electrode site of the electrode site is 10 microns to 10 millimeters, and a thickness of the electrode site is 5nm to 200μm.

8. The flexible electrode according to claim 1 or 2, wherein a contact surface of the electrode side for contacting a biological tissue has a surface modification layer for improving an electrochemical property of the electrode site.

9. The flexible electrode according to claim 8, wherein any one or more of a conductive polymer and conductive metal particles are used for surface modification, the conductive polymer comprises poly(ethylenedioxythiophene), poly(p-styrenesulfonic acid), polypyrrole, and a material of the conductive metal particles comprises iridium, iridium oxide, platinum, and platinum iridium alloy.

10. The flexible electrode according to claim 1 or 2, further comprising a rear end portion, wherein the implanted portion extends from the rear end portion, and the rear end portion comprises a rear end site coupled to one of the wires in the wire layer and a rear end circuit, to achieve signal transmission between the electrode site electrically coupled to one of the wires and the rear end circuit.

11. The flexible electrode according to claim 10, wherein the rear end site is located in the wire layer and is exposed via a through-hole in at least one layer of the first insulating layer and the second insulating layer.

12. The flexible electrode according to claim 10, wherein the rear end site is located between the wire layer and one layer of the first insulating layer and the second insulating layer, and is exposed via a through-hole in another layer of the first insulating layer and the second insulating layer.

13. The flexible electrode according to claim 12, wherein the rear end site comprises a conductive sub-layer, and a material of the conductive sub-layer is any one of gold, platinum, iridium, tungsten, magnesium, molybdenum, platinum-iridium alloy, titanium alloy, graphite, carbon nanotubes, PEDOT, or a combination thereof.

14. The flexible electrode according to claim 12, wherein a thickness of the rear end site is 5 nanometers to 200 micrometers.

15. The flexible electrode according to claim 12, wherein the rear end site further comprises an adhesion sub-layer close to the wire layer, and a material of the adhesion sub-layer is any one of chromium, tantalum, tantalum nitride, titanium, titanium nitride, or a combination thereof.

16. The flexible electrode according to claim 1 or 2, wherein the wire layer comprises a conductive sub-layer, and a material of the conductive sub-layer is any one of gold, platinum, iridium, tungsten, platinum-iridium alloy, titanium alloy, graphite, carbon nanotubes, PEDOT, or a combination thereof.

17. The flexible electrode according to claim 16, wherein a thickness of the conductive sub-layer is 5 nanometers to 200 micrometers.

18. The flexible electrode according to claim 10, wherein the wire layer comprises a conductive sub-layer and an adhesion sub-layer close to any one of the electrode site and the rear end site, and a material of the adhesion sub-layer is any one of chromium, tantalum, tantalum nitride, titanium, titanium nitride, or a combination thereof.

19. The flexible electrode according to claim 1 or 2, wherein a cross-sectional area of the wire is 0.01 square micrometers to 1 square millimeter.

20. The flexible electrode according to claim 1 or 2, wherein the first insulating layer and the second insulating layer have a thickness of 0.5 micrometers to 1 millimeter.

21. The flexible electrode according to claim 1 or 2, wherein a material of the first insulating layer and the second insulating layer is any one of polyimide, polydimethylsiloxane, parylene, epoxy resin, polyamide imide, SU-8 photoresist, silica gel, silicone rubber, or a combination thereof.

22. The flexible electrode according to claim 1 or 2, wherein a material of the wire layer is any one of magnesium, molybdenum and an alloy thereof, or a combination thereof, and a material of the first insulating layer and the second insulating layer is any one of polylactic acid and polylactic acid-glycolic acid copolymer, or a combination thereof, so that the flexible electrode is biodegradable.

23. The flexible electrode according to claim 1 or 2, wherein the electrical stimulation is a long-term electrical stimulation or a short-term electrical stimulation.

24. The flexible electrode according to claim 1 or 2, wherein the identical or different electrical stimulations include currents or voltages with identical or different polarities.

25. The flexible electrode according to claim 1 or 2, wherein the identical or different electrical stimulations include currents or voltages with identical or different amplitudes, wave widths, and frequencies.

26. The flexible electrode according to claim 1 or 2, wherein the implanted portion has an installation hole through which an electrode implantation device is attached to the flexible electrode to perform implantation.

27. The flexible electrode according to claim 10, wherein after separating the flexible electrode from a substrate, the rear end portion is connected to the rear end circuit, and the rear end portion and the rear end circuit are packaged together by any one of epoxy resin and polydimethylsiloxane, or a combination thereof.

28. A method for manufacturing a flexible electrode for acupuncture according to any one of claims 1 to 27, comprising:
forming the first insulating layer, the wire layer, the second insulating layer, and the electrode site on a substrate; and
separating the flexible electrode from the substrate,
wherein a through-hole is manufactured by patterning at a position corresponding to the electrode site in at least one layer of the first insulating layer and the second insulating layer.
